(19) United States
(12) Patent Application Publication
(10) Pub. No.: US 2020/0170998 A1
(43) Pub. Date: Jun. 4, 2020

(54) UMBILICAL CORD BLOOD SERUM AND UMBILICAL CORD BLOOD PLASMA PREPARATION IN COMBINATION WITH A NONSTEROIDAL ANTI-INFLAMMATORY DRUG AND FREEZE-DRYING THE COMPOSITION

(71) Applicant: CELLULA LLC, Villanova, PA (US)

(72) Inventors: Damian Joseph Todd, Villanova, PA (US); Dennis Todd, Tuxedo Park, NY (US)

(73) Assignee: CELLULA LLC, Villanova, PA (US)

(21) Appl. No.: 16/698,896

(22) Filed: Nov. 27, 2019

Related U.S. Application Data
(60) Provisional application No. 62/772,836, filed on Nov. 29, 2018.

(51) Int. Cl. A61K 31/407; A01N 1/02; A61K 9/00; A61K 35/51; A61K 31/573; A61K 9/19; A61K 45/06

(52) U.S. Cl. CPC A61K 31/407; A01N 1/0221; A61K 9/0048; A61K 45/06; A61K 31/573; A61K 9/19; A01N 1/0226; A61K 35/51

(57) ABSTRACT

A process to prepare and freeze-dry umbilical cord blood serum and/or umbilical cord blood plasma with a nonsteroidal anti-inflammatory drug and/or ophthalmic steroids. The freeze dried composition may then be administered using a system for delivering predetermined volumes of the compound to the eye or other parts of the mammalian body.

### CROSS REFERENCE TO RELATED APPLICATION

This application claims the benefit of U.S. Provisional Patent Application Ser. No. 62/772,836, filed Nov. 29, 2018, which is incorporated by reference herein as if fully set forth.

### SUMMARY

This disclosure broadly covers a method for preparing Umbilical Cord Blood serum (UCBs) and/or Umbilical Cord Blood plasma (UCBp) and then freeze-drying the serum or plasma along with a nonsteroidal anti-inflammatory drug (NSAID), and or ophthalmic antibiotics, and or ophthalmic steroids. This disclosure describes the process for preparing UCBs and the process for freeze-drying the product. This disclosure details two processes for collecting UCB and preparing serum, one method for the conversion of UCBp to serum and the method for freeze-drying the UCBs NSAID/ophthalmic antibiotic/ophthalmic steroid product. This disclosure describes the process for preparing UCBp and the method for freeze-drying the UCBp. The resulting preparations have application in performing treatments of the eye, ear, nose or other parts of a body with UCBs and freeze-dried UCBs and freeze-dried UCBp. Current cord blood treatments do not involve freeze-drying UCBp or UCBs.

The resulting product from may be used in conjunction with a single-dose dual-compartment drug delivery system to treat the eye or other parts of the mammalian body. An example of a single-dose dual-compartment drug delivery system is described in U.S. Provisional Application No. 62/629,624 and U.S. patent application Ser. No. 16/274,163, the contents of which are hereby incorporated by reference. This device may also be constructed in a similar fashion to a glow stick commonly sold on the commercial market but with an applicator attachment.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 is a diagram of the single-use, disposable applicator according to one embodiment of this disclosure; and

FIG. 2 is an illustration of the barrier shown in FIG. 1, according to one embodiment of this disclosure.

### DETAILED DESCRIPTION

In one embodiment, a freeze-dried composition including allogeneic serum and an antibiotic and/or a nonsteroidal anti-inflammatory drug (NSAID) and/or an ophthalmic steroid is disclosed. A method of treating ophthalmic diseases (e.g., ocular burns, thermal and chemical, ocular GVHD, and dry eye syndrome) with this freeze dried composition is also disclosed.

The allogeneic serum of the freeze-dried composition may be derived from, but not limited to, umbilical cord blood (UCB).

The antibiotics of the freeze-dried composition may be of any antibiotic manufactured for ophthalmic use and proven to have appropriate antibiotic properties. These antibiotics include but are not limited to tobramycin, gentamicin, ciprofloxacin, norfloxacin, or bacitracin.

The NSAID of the freeze-dried composition may be any NSAID manufactured for ophthalmic use (e.g., Ketorolac) and proven to have analgesic and/or anti-inflammatory activity.

The freeze-dried ophthalmic steroid may be of any steroid manufactured for ophthalmic use (e.g., fluorometholone, dexamethasone, prednisolone, etc.). Ophthalmic steroids are used in managing postoperative inflammation following various ocular surgeries, anterior uveitis, ocular allergies, external eye inflammatory diseases associated with infections, corneal injury from chemical, radiation or thermal burns and penetration of foreign bodies. These ophthalmic steroids include, but are not limited to: Maxidex® [package insert], Fort Worth (Tex.): Alcon Laboratories Inc.; 2007 May, Dexamethasone sodium phosphate solution [package insert], Fort Worth (Tex.): Falcon Pharmaceuticals, Ltd.; 2006 March, Durezol® [package insert], Tampa (Fla.): Fort Worth (Tex.): Alcon Laboratories Inc.; 2013 May, Flarex® [package insert], Fort Worth (Tex.): Alcon Laboratories Inc.; 2006 December, Fluor-Op® [package insert], Duluth (Ga.): Novartis Ophthalmics; 2006 May, FML® ointment [package insert], Irvine (Calif.): Allergan Inc.; 2007 October, FML® suspension [package insert], Irvine (Calif.): Allergan Inc.; 2003 June, FML Forte® [package insert], Irvine (Calif.): Allergan Inc.; 2004 June, Alrex® [package insert], Tampa (Fla.): Bausch & Lomb Inc.; 2013 August, Lotemax® gel [package insert], Tampa (Fla.): Bausch & Lomb Inc.; 2014 October, Lotemax® ointment [package insert], Tampa (Fla.): Bausch & Lomb Inc.; 2011 April, Lotemax® suspension [package insert], Tampa (Fla.): Bausch & Lomb Inc.; 2013 August, Omnipred® [package insert], Fort Worth (Tex.): Alcon Laboratories Inc.; 2007 October, Pred Forte® [package insert], Irvine (Calif.): Allergan Inc.; 2013 August, Pred Mild® [package insert], Irvine (Calif.): Allergan Inc.; 2014 April, Prednisolone sodium phosphate solution [package insert], Tampa (Fla.): Bausch & Lomb Inc.; 2013 January, Vexol® [package insert], Fort Worth (Tex.): Alcon Laboratories Inc.; 2008 March.

The use of serum-based eye drops prepared from UCB has been proposed to treat various intractable ocular conditions. Studies have demonstrated that the protein profile of UCBp is similar to that of human plasma and similar to human tears (for example, Yoon K C, et al., ARVO Annual Meeting Abstract, May 2005).

UCB is a readily available product collected frequently for its hematopoietic stem cell content. After the stem cells have been isolated, the remaining plasma is typically discarded. The resultant plasma may be subsequently converted to serum. Alternatively, serum may be obtained directly by collecting non-anticoagulated UCB.

Sharma et. al. reported on a study utilizing UCBs eye drops for severe ocular chemical burns (Sharma N, et.al., Invest Ophthalmol Vis Sci. 2011 Feb. 25; 52(2):1087-92). There were 32 patients divided into one of three groups: UCBs eye drops, autologous serum eye drops, and artificial tears (AT). They concluded that UCBs therapy was more effective in treating chemical burns then either autologous serum eye drops or AT. Mean time to complete epithelialization was 21.16±26.81, 56.6±35.5, and 40.13±35.79 days, respectively.

In a later study, Sharma et. al. compared the efficacy of amniotic membrane transplantation (AMT) with UBCs in 55 eyes with grade III, IV and V chemical burns (Sharma N, et al., Am J Ophthalmol. 2016; 168:157-163). 20 eyes received conventional medical management alone or combined with UCBs (17 eyes) or AMT (18 eyes). UCBs and AMT groups showed early epithelialization compared to the conventional medical management group. Meantime of the healing of epithelial defect was 57.7±29.3, 27.4±19.0, 44.1±28.9 days in the conventional medical management, UCBs, and AMT groups respectively (p=0.02).

Oh et al. studied the effect of UCBs eye drops on corneal wound healing in ocular burns compared with peripheral blood serum (PBS) eye drops or AT in 6 eight-week old mice (Oh et al., Curr Eye Res. 2012 December; 37(12):1084-90). The UCBs group showed lower epithelial defect parameters compared with the PBS group at 1 and 2 days (p<0.05), and with the AT group from 1-5 days (p<0.05). The haze scores were significantly lower in the UCBs group than in the PBS group at 2 and 3 days (p<0.05), and in the AT group from 2-7 days (p<0.05). Histological examination showed better epithelial integrity and lower stromal inflammation and edema in the UCBs group than the other groups. IL-1β levels were 99.71±85.22 and 230.76±102.67 pg/mL in the UCBs and PBS groups, respectively (p=0.03). UCBS eye drops were shown to be more effective in improving corneal wound healing and reducing corneal haze compared with PBS eye drops and AT in experimental chemical burns.

Sharifi et al. investigated the efficacy of fetal human UCBss on healing of alkali corneal damage in a rabbit model. (Sharifi B, et al. Life Science Journal 2013; 10(7s): 731-4). The study involved 16 rabbits, 32 eyes randomized between human fetal UCBSs and AT treatments. The average wound healing rate in the intervention group based on of defect diameter was 0.77(SD=0.013) mm/day and the control group was 0.73 (SD=0.018) mm/day. This study showed that fetal human UCBs compared with AT increased rate of corneal alkali wound healing in the rabbit model. More evaluation based on UCBs treatment was suggested.

Yoon et al. reported on a limited study of 12 bone marrow transplant patients with severe dry eye syndrome associated with ocular graft vs Host Disease (oGVHD) (Yoon K C, et al., Bone Marrow Transplant. 2007 February; 39(4):231-235). These patients were refractory to conventional treatments. Six months after treatment, significant improvement was observed in symptom score (from 3.83+/−0.38 to 0.83+/−0.57, P<0.01), corneal sensitivity (from 52.08+/−6.06 mm to 57.50+/−3.00 mm, P<0.01), tear film BUT (from 2.50+/−0.91 s to 5.71+/−1.04 s, P<0.01), and keratoepitheliopathy score (from 7.42+/−2.02 to 1.29+/−0.46, P<0.01). There was no significant change in Schirmer test and TCR results. No significant complications associated with the use of the eyedrops were observed. Yoon et. al. concluded that UCBs eye drops are safe and may be an effective way to treat severe dry eye associated with oGVHD.

While current data on cytokine, chemokine and growth factor concentrations in UCB is limited, a study by Oh et al. has shown that factors present within UCBs contribute to improved epithelial healing for chemical ocular burns in a mouse model when compared to peripheral blood serum (PBS) (Oh et al., Current Eye Research, Volume 37, 2012 Issue 12). Within this study and others, UCBs have been shown to contain biological factors that promote epithelial growth such as EGF, FGF, PGF, neurotrophic factors such as substance P, TGF-β, IGF-1, NGF and tear components such as vitamin A and fibronectin. These factors are believed to contribute to the epithelial healing capabilities of UCBs. In this same study, Oh demonstrated that the concentrations of EGF, TGF-β, and NGF are higher in UCBs than PBS and that UCBs eye drops and were, in fact, superior to PBS eye drops in promoting corneal epithelial healing.

Although the use of UCBS eye drops has been studied in several small clinical trials, the exact mechanism of action, as stated previously, is unclear. Choi proposed that serum growth factors and anti-inflammatory cytokines are important in corneal healing (Choi et.al, Molecular Vision 2009: 15:2230-2238). Their in-vitro study involved amniotic membrane lysates and suggests that EGF, KGF, HGF, and FGF are key biochemical factors in ocular surface re-epithelization.

Antibiotics may be used to treat or prevent bacterial infections. Due to the conditions in which an ocular abrasion or burn may occur, infection is a distinct possibility. Therefore, the addition of an antibiotic commonly used in ophthalmic treatments will be of benefit to a patient. These antibiotics include, but are not limited to, tobramycin, gentamicin, ciprofloxacin, norfloxacin, and/or bacitracin.

Topical nonsteroidal anti-inflammatory drugs (NSAIDs) are used to control pain and postoperative inflammation. All NSAIDs are associated with some adverse effects. Lee studied the toxicity of three NSAIDs along with one steroidal agent on corneal epithelial cells in-vitro. (Lee et.al., J Korean Med Sci, 2015; 30:1856-1864). They showed that the steroid, Flourometholone, induced the most severe toxicity. Diclofenac blocks the lipoxygenase pathway at high concentrations along with lower Na+ concentrations and higher amounts of preservatives leading to significant cellular toxicity. Both bromfenac and pranoprofen showed less cellular damage and showed no difference with the control, a balanced salt solution. With the use of NSAIDS in topical ophthalmic treatment of patients, surface toxicity has been a concern, and incidents of keratitis, corneal subepithelial infiltrates, ulceration, and corneal melts have been reported (Guidera et.al., Ophthalmology, 2001, 108 (5), pp. 936-944; Solomon et al, J Cataract Refract Surg, 2001, 27 (8), pp. 1232-1237; Teal et al, J Cataract Refract Surg, 1995, 21(5), pp. 516-518). Further, patients often report burning or stinging on instillation (Jaanus et.al., Anti-Inflammatory Drugs. Clinical Ocular Pharmacology, Bartlet, J. D. and Jaanus, S. D., Ed., Boston: Heineman, 2001, pp. 265-298). The burning or stinging could be related to the concentration of the active component of the formulation.

Ketorolac tromethamine 0.5% (w/v) ophthalmic solution, available from Allergan, Inc. under the trade name Acular®, is a safe and effective NSAID with proven analgesic and anti-inflammatory activity. The most common adverse events associated with the use of the 0.5% ketorolac formulation is ocular irritation, primarily burning and stinging on instillation. Eliminating or reducing ocular irritation has the potential for improving tolerability, compliance, and effectiveness of treatment. Ketorolac has the following chemical structure.

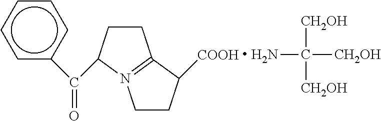

It was discovered that reducing the concentration of ketorolac tromethamine in the ophthalmic solution reduces the occurrence of adverse events while maintaining clinical efficacy. This led to an aqueous topical ophthalmic composition comprising from 0.35% to 0.45% ketorolac tromethamine.

In one embodiment, a method of treating an individual suffering from an ocular burn, thermal or chemical, or oGVHD includes the administration to the patient of a sterile composition comprising from 0.35% to 0.45% weight to volume of ketorolac tromethamine or similar NSAID with umbilical cord blood serum. The umbilical cord blood serum in this embodiment is lyophilized and reconstituted.

In one embodiment, a method of treating or preventing ocular pain in an individual suffering from an ocular burn, thermal or chemical, or oGVHD includes the administration to the patient a sterile composition comprising an ophthalmic steroid of 0.1%-0.5% weight to volume and umbilical cord blood serum. The umbilical cord blood serum in this embodiment is lyophilized and reconstituted.

In one embodiment, an ophthalmic eye drop is composed of freeze-dried allogeneic serum, e.g., UCB derived, and an ophthalmic NSAID, similar to, but not restricted to, Ketorolac Tromethamine, or an ophthalmic steroid similar to, but restricted to, prednisolone sodium phosphate. All of the aqueous topical ophthalmic compositions of this disclosure are contemplated for use in treating ocular burns, thermal and chemical, acute and chronic GVHD, and/or postoperative treatment following ocular surgeries.

Dry eye syndrome represents a serious complication of GVHD, Sjögren syndrome, rheumatoid arthritis, and collagen vascular diseases. This may result in the inability to produce tears. When tears do not adequately lubricate the eye, a person may experience pain, light sensitivity, a gritty sensation, a feeling of a foreign body or sand in the eye, itching, redness, and/or blurring of vision. Left untreated, the condition could lead to corneal ulceration and blindness. UCB/NSAID/ophthalmic steroid based eye drops can be used to treat severe dry eye but also ocular injuries sustained from chemical and thermal burns as well as foreign bodies in the eye.

Current UCB based treatment options have limited shelf-life and must be stored under refrigerated conditions, limiting their application. Lyophilizing the composition will stabilize the product allowing for storing the composition under varying environmental conditions.

According to embodiments of this disclosure, umbilical cord blood serum is prepared. First, serum and plasma is collected. Human umbilical cord blood serum and plasma are collected from volunteer donor mothers during vaginal or cesarean section delivery after signing an Informed Consent administered by a trained healthcare professional. The Informed Consent must be reviewed and approved by a registered Institutional Research Board (IRB). Whole blood specimens should ideally be processed in less than 2 hours after collection is completed, but may be processed up to 8 hours after collection.

Voluntary umbilical cord blood donors will undergo a physical examination and screening to detect the presence of past or present infections in accordance with FDA and AABB guidelines. Infectious disease testing will include: Hepatitis B Surface Antigen, Hepatitis B Core Antigen, Hepatitis B Virus NAT, Hepatitis C Virus Encoded Antigen, Hepatitis C Virus NAT, HIV-½ NAT, Human T-Lymphotropic Virus Types I and II NAT, Treponema pallidum (Syphilis), Trypanosoma cruzi (T. cruzi), Babesia, West Nile Virus, Zika Virus, and CMV (Cytomegalovirus).

Should the donor mother test positive for any of the infectious disease markers listed above, the umbilical cord blood serum must be destroyed to prevent the possibility of transmitting the disease for which the mother tested positive.

After the delivery of the infant, the healthcare professional should double clamp the cord on the baby side. The placenta is then elevated. The healthcare professional will cleanse a 4″-6″ area along the umbilical vein with alcohol followed by betadine to remove maternal blood and contaminants. The healthcare professional will always clean the area in a single direction taking care not to wipe over the area with the same swab. The healthcare professional will insert the needle into the umbilical vein holding the collection needle at downward angle with the bevel side up following the direction of the vein, avoiding puncturing through the vein. The healthcare professional will collect cord blood in an SST or NON (red top) tube(s) used for serum collection. When the collection is complete, the healthcare professional will package the collection tubes filled with the umbilical cord blood and the completed donor documents along with the blood collection tubes from the donor mother and ship to the laboratory for processing.

The healthcare professional will place the tubes in a 4° C. refrigerator 1 to 4 hours and allow the blood to clot. Next, the healthcare professional will remove the tubes containing the clotted cord blood from the refrigerator and centrifuge the tubes at 2500 rpm for 10 minutes at RT. The serum is removed from the clot by gently pipetting off the supernatant (serum) into a clean tube using a sterile glass pasteur pipette. The serum is re-centrifuged to ensure all cellular debris is removed. The serum is placed into 5 Dram (20 ml) clear Borosilicate Glass Screw-Top Vials (Black Phenolic Cap w/Rubber Liner) freezing vial(s), and the vial(s) are labeled with the total bleed and date, and stored at −20° C.

Umbilical cord blood plasma collection will be described. After the delivery of the infant, the healthcare processional will clamp the umbilical cord, and double clamp the cord on the baby side. The placenta is then elevated. The healthcare processional will cleanse a 4″-6″ area along the umbilical vein with alcohol followed by betadine to remove maternal blood and contaminants. The healthcare processional will always clean the area in a single direction taking care not to wipe over the area with the same swab. Using an FDA approved blood collection bag containing an anticoagulant, the healthcare processional will insert the needle into the umbilical vein holding the collection needle at downward angle with the bevel side up following the direction of the vein, avoiding puncturing through the vein. The healthcare processional will allow the blood to drain into the collection bag, gently rocking the bag to allow thorough mixing of the blood with the anticoagulant. When the collection is complete, the healthcare processional will package the blood bag, the Vacutainer tubes from the mother and the completed donor documents and ship to the processing laboratory.

Whole blood specimens should ideally be processed in less than 2 hours after collection is complete but may be processed up to 8 hours after collection.

When the blood arrives in the processing laboratory, the tubes and collection bag are inspected and the donor documents and the donor identification on the blood collection tubes and blood bag are reviewed to ensure that the information is complete. The contents of the collection bag are transferred to a centrifuge and spun at 1,200×g for 10 minutes. When centrifugation is complete, a worker will transfer the blood bag to a plasma expresser. The worker will then express the plasma into the secondary bag being careful not to allow any cells or platelets to enter the secondary bag. The worker seals the tubing between the collection bag and the secondary bag and separates the two. The worker labels the secondary bag with the appropriate donor ID. Finally, the worker will forward the donor mother's blood tubes for infectious disease marker analysis, as described above.

Conversion of Umbilical Cord Blood Plasma to Serum will now be described. A worker will determine the volume of plasma to be converted. Plasma is then incubated for 30 minutes at 37° C. To the incubated plasma, 100 μL of thrombin is added per 10 mL of plasma. For example, for 50 mL of plasma add 500 μL thrombin solution. The thrombin/plasma mixture is incubated for 10 minutes at 37° C. The mixture is then allowed to clot at room temperature for 1 hr. After clotting, samples are frozen at −20° C. for 2 to 4 hrs. Samples are then thawed at RT and 1 g of Kaolin is added per each 10 mL of plasma. The samples are then mixed continuously for 4 hrs and incubated at 2 to 8° C. overnight with gentle rocking. Next, the samples are centrifuged at 1,935×g for 45 minutes. The supernatant is decanted into centrifuge tubes. The resulting serum is filtered through 0.45 and 0.22 μm pore size filter membranes.

Freeze-Drying Umbilical Cord Blood Serum is now described. Lyophilization or freeze-drying is a process in which water is removed from a product after it is frozen and placed under a vacuum, allowing the ice to change directly from solid to vapor without passing through a liquid phase. This process is known as sublimation. The process consists of three separate, unique, and independent processes: freezing, primary drying (sublimation), and secondary drying (desorption).

The advantages of lyophilization include: enhanced product stability in a dry state, removal of water without excessive heating of the product, and rapid dissolution of the product with an appropriate diluent.

The process involves the following steps: sterilizing the serum by passing it through a 0.22 micron bacteria-retentive filter. Filling the sterilized serum into a sterile container and partially stoppering the container under aseptic conditions, placing the container in the lyophilizer chamber under sterile conditions, freezing the serum in the lyophilizer chamber or freezing the serum in another chamber, applying a vacuum to the chamber and heating the shelves in order to evaporate the water from the frozen state, and, finally, stoppering the vials by a hydraulic or screw rod stoppering mechanism installed in the lyophilizer. (Lyophilization of Parenterals (7/93) FDA Guidance, Guide to Inspections of Lyophilization of Parenterals). The NSAID, and/or steroid, and/or antibiotic are added before lyophilization to the cord blood serum. Alternatively, the NSAID, and/or steroid, and/or antibiotic can be added after lyophilization or after reconstitution of the cord blood serum as a component of the reconstitution fluid.

The embodiments described herein may be used as a process for preparing UCBs coupled with an NSAID or ophthalmic antibiotic or ophthalmic steroid, freeze-drying the product for medical application in a variety of environments. The resulting lyophilized product is advantageous over current treatments because the freeze-drying eliminates the need to store the product in a freezer and extends the product's usable life. Freeze drying also enables an improved shipping experience.

The aforementioned lyophilized product may be used in combination with the single-dose dual-compartment drug delivery system (described in U.S. patent application Ser. No. 16/274,163, incorporated by reference). In one embodiment, the drug delivery system includes two separate compartments in a single use applicator, each of the two separate compartments being separated by a barrier or membrane which keeps the materials in the two compartments separate until pressure is applied to the system. Once pressure is applied to the system, for example, by way of squeezing the system, the barrier or membrane will break apart and the materials in the two compartments will mix, producing the final product to be applied from the system. This system, also referred to herein as an applicator, has application in performing treatments of the eye, ear, nose or other parts of a body in a single dosage and in a reliable manner.

Current applicators do not have the ability to serve as a single dosage, disposable applicator that allows two separate ingredients to be mixed at the time of the users' choosing. Prior art delivery systems fail to provide a system for mixing a liquid component and a powder component, in the manner described above, for application at a time of the user's choosing. The single-dose applicator may be disposable, and also allows for extended shelf life of the medication by keeping the unmixed ingredients in a sterile, unmixed state until ready for use.

The single use applicator described herein allows for simplicity while ensuring that only the necessary amount of product in each compartment is utilized when needed. The inventive drug delivery device comprises a housing with two compartments, each containing substances separated by a barrier or membrane which can be broken apart by applying pressure to the housing. One end of the applicator comprises a tapered nozzle and cap which can be twisted off when ready to use. Once the barrier or membrane is broken, the two components, liquid and powder, will mix creating the end product. After the two components have been mixed and the user is ready to apply the end product, the user will twist the cap off of the device and apply pressure to the applicator housing in order to force the substance through the nozzle and into the eye, nasal passage, ear canal or other part of the body.

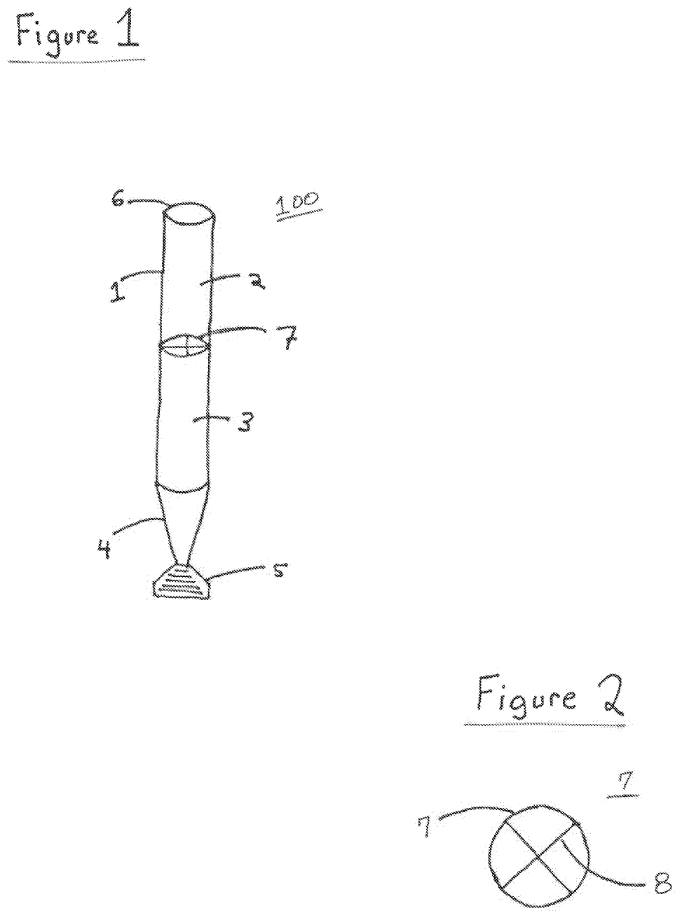

Referring to FIG. 1, one embodiment of the disclosed applicator 100 is shown. While the applicator 100 is depicted as cylindrical in this embodiment, that is purely exemplary. Various shapes may be utilized for both ease of manufacturing and for surface area and mixing considerations of the components. The applicator 100 comprises a housing 1. In some embodiments, the housing 1 may be made from a firm plastic material or a rubber or resin material. Preferably, the housing 1 is constructed such that it is malleable enough that when pressure is applied by the fingers of a user of the applicator 100, a barrier 7 is ruptured or broken. This will be described in more detail below.

The housing 1 forms two compartments, the first compartment 2 and the second compartment 3. In one embodiment, the first compartment 2 contains a dry component, and the second compartment 3 contains a liquid component (although obviously this may be switched). The volume of these compartments may be changed based on the desired ratio of components to be mixed. Similarly, the shape of the housing 1 forming the first compartment 2 and second compartment 3 may be selected to increase or decrease the size of the interface between the first compartment 2 and second compartment 3. For example, the cylindrical shape of the housing 1 shown in FIG. 1 provides a relatively small interface, in terms of surface area, between the first compartment 2 and the second compartment 3. Instead of a cylinder, in another embodiment, the housing 1 is spherical, thus maximizing the surface area of the interface between a first compartment 2 and a second compartment 3. This size of this interface may be set as desired based on the solubility of the components to be mixed.

The housing 1 further comprises an applicator nozzle 4 and an applicator cap 5 on one end, and a base 6 at the other end. The applicator cap 5 and the base 6 may be affixed to the housing via screw threads, or they may be sealed plastic during manufacture. The applicator cap 5 and the base 6 serve to seal the applicator 100. During manufacture, components may be added to first compartment 2 and sealed by the base 6. Similarly, components may be added to the second compartment 3 and sealed by the applicator cap. The applicator nozzle 4 may be shaped to allow for proper application of the mixed solution. For example, the shape and opening of the applicator nozzle 4, once the applicator cap 5 has been removed, may produce a desired droplet size and/or shape. Additionally, the shape and contour of the applicator nozzle 4 may be selected to aid in the administration of the mixed solution, for example, depending on whether the mixed solution is to be applied to an eye, or to an ear, etc.

Still referring to FIG. 1, the first compartment 2 and the second compartment 3 of the applicator 100 are separated by barrier 7. Barrier 7 separates the components containing in the first compartment 2 and the second compartment 3. The barrier 7 is not permeable. However, barrier 7 is constructed such that when pressure is applied to the housing, for example by way of the user of the applicator 100 squeezing the housing 1 with their fingers, the pressure created by the first compartment 2 and/or the second compartment 3 ruptures the barrier 7 material 7. This rupture of the barrier 7 allows the contents of the first compartment 2 to mix with the contents of the second compartment 3. As mentioned above, in one embodiment, the dry component and liquid component mix after the barrier 7 has ruptured.

Referring now to FIG. 2, the barrier 7 is shown. It is important to note that the shape of the barrier 7 is exemplary. Since the applicator 100 described with reference to FIG. 1 is cylindrical, the barrier 7 is a cross section of the housing, and is therefore circular in shape. As mentioned above, the barrier 7 may be any shape selected for the specific components housed in the applicator 100. The barrier 7 may be constructed from the same material as the housing 1 described in FIG. 1, or it may be made of a different material. The barrier 7, in one embodiment, includes seams 8. The purpose of the seams 8 is to enable rupture of the barrier 7 when pressure is applied to the housing. Depending on the material of the barrier 7, the seams 8 may not be necessary. When the seams 8 are necessary, they may be arranged in a variety of ways selected such that the barrier 7 ruptures when the desired force is applied to the housing 1. FIG. 2 shows the seams 8 in an “X” arrangement, but this is exemplary.

In one embodiment, the applicator 100 is used as a single-use, disposable eye dropper to deliver umbilical cord blood derived serum to a patient's eye. This may be for the treatment of thermal and/or chemical burns or for the treatment of dry-eye syndrome, as described above. The umbilical cord blood is processed to extract the umbilical cord blood serum as described above. The umbilical cord blood serum is then freeze dried, resulting in a powder. In embodiments, an NSAID, a steroid, and/or an antibiotic is a component of the powder. The freeze dried umbilical cord blood serum powder is placed in one compartment of the applicator 100. A sterile liquid solution is placed in the other compartment of the applicator 100. The sterile liquid solution may be sterile water, or it may be some other liquid solution. When pressure is applied to the housing 1 of the applicator 100, for example, by the user of the applicator 100 squeezing the applicator 100 between their fingers, the barrier 7 ruptures, permitting mixing of the freeze dried umbilical cord blood serum in one compartment and the sterile liquid solution in the other compartment. The user of the applicator 100 may then shake the solution as necessary to encourage mixing of the components. The applicator cap 5 can then be removed by the user, and the reconstituted umbilical cord blood serum may be applied to the user's eye in a typical eye dropper fashion.

In other embodiments, a single use, disposable eye dropper operates in a similar fashion as a glow stick. In this embodiment, an outer vessel houses and inner vessel. The inner vessel includes the freeze dried umbilical cord blood powder including a NSAID, and/or a steroid, and/or an antibiotic as described above. The outer vessel houses a sterile liquid solution. When the glow stick, i.e. the outer vessel, is bent, the inner vessel, which is made of a brittle material, ruptures allowing the contents to mix. The out vessel has an applicator at one end that is then use to administer the reconstituted umbilical cord blood serum eye drops including a NSAID, and/or a steroid, and/or an antibiotic. In an alternative embodiment, the inner vessel contains the sterile solution and the outer vessel contains the umbilical cord blood powder including a NSAID, and/or steroid, and/or antibiotic.

What is claimed is:

1. A composition for the treatment of ocular burns, the composition comprising:
lyophilized umbilical cord blood serum; and
a non-steroidal anti-inflammatory drug (NSAID),
wherein the composition is reconstituted by combination with a sterile solution.

2. The composition of claim 1, further comprising:
an antibiotic for ophthalmic use.

3. The composition of claim 2, wherein the antibiotic for ophthalmic use is selected from the group consisting of tobramycin, gentamicin, ciprofloxacin, norfloxacin, and bacitracin.

4. The composition of claim 1, wherein the NSAID is ketorolac tromethamine.

5. The composition of claim 4, wherein the ketorolac tromethamine comprises 0.35% to 0.45% weight to volume of the composition.

6. The composition of claim 1, wherein the NSAID has the following chemical structure:

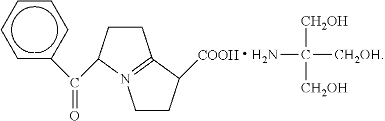

7. The composition of claim 1, further comprising:
a ophthalmic steroid.

8. A composition for the treatment of ocular burns, the composition comprising:
lyophilized umbilical cord blood serum; and
the non-steroidal anti-inflammatory drug (NSAID) ketorolac tromethamine in an amount between 0.35% to 0.45% by weight,
wherein the composition is reconstituted by combination with a sterile solution.

9. A method of treating a patient having an ocular burn, the method comprising:
administering a therapeutic amount of the composition of claim 8.
